(19) United States
(12) Patent Application Publication
(10) Pub. No.: US 2002/0004656 A1
(43) Pub. Date: Jan. 10, 2002

(54) Disposable diaper system

(76) Inventors: Farzana Khan, Lanham, MD (US); Jacquel M. Smith, Fort Washington, MD (US)

Correspondence Address: Richard C. Litman, LITMAN LAW OFFICES, LTD., P.O. Box 15035, Arlington, VA, 22215, US

(21) Appl. No.: 09/861,613

(22) Filed: May 22, 2001

Related U.S. Application Data
(60) Provisional application No. 60/216,701, filed on Jul. 7, 2000.

(51) Int. Cl. A61F 13/15; A61F 13/20

(52) U.S. Cl. 604/385.06; 604/385.13

(57) ABSTRACT

A disposable diaper system for infants, babies, senior citizens and/or those who are physically challenged is presented. The system includes a diaper formed as a substantially rectangular blank having tabs at the corners and elastic leg engaging areas to prevent or minimize leakage and to provide a secure and comfortable fit. For hygiene purposes the system includes a first and second storage compartment coextensive therewith for storing an integrated disposable bag and gloves for reducing feces and/or body fluid contamination during diaper removal. The system is configured with materials which provide a reduced overall package volume including both the bag and gloves.

### CROSS-REFERENCE TO RELATED APPLICATION

This application claims the benefit of U.S. Provisional Patent Application Serial No. 60/216,701, filed Jul. 7, 2000.

### BACKGROUND OF THE INVENTION

1. Field of the Invention

The present invention relates generally to diapers. More specifically, the invention is an improved combination disposable diaper system or kit for discarding disposable diapers without feces and/or body fluid contamination.

2. Description of Related Art

Numerous diaper systems have been devised which integrate the use of a disposable bag for easy removal and discarding of soiled diapers from babies. However, one of the most troublesome occurrences with diaper changing is having to seek a means for decontaminating one's hands and not find anything sufficient to do so after removing a soiled or otherwise disposable diaper. Also, it seem as if the stench lingers long after a suitable cleaning source has been found.

This, particularly, for the baby care taker causes or translates into other difficulties, such as overloaded carry bags which hold any number of custodial items for taking care of the needs of an infant or baby. As clearly identified in the following U.S. patents issued to Schaar U.S. Pat. Nos. 3,731,689, 4,034,760, Gellert U.S. Pat. No 4,085,753, Izzo U.S. Pat. No 4,493,713, Dean et al. U.S. Pat. No 4,923,455, Barrett U.S. Pat. No 5,141,505 and Powell U.S. Pat. No 4,743,240, conventional bag type diaper systems having the aforementioned deficiencies are well known.

What is needed, however, is a disposable diaper system which integrates a combination diaper, disposable bag and a glove kit in order to eliminate the problems of conventional diaper systems.

Other U.S. patents such as those issued to Azpiri U.S. Pat. No 4,430,087, Schaar U.S. Pat. No 3,927,674 and Lerner U.S. Pat. No 4,788,733 disclose conventional diaper features and auxiliary cleaning features which are considered to be of general relevance to the disposable diaper system as herein described. Thus, none of the above inventions and patents, taken either singly or in combination, is seen to describe the instant invention as claimed.

### SUMMARY OF THE INVENTION

The disposable diaper system for babies, senior citizens and/or the physically challenged according to the invention includes a diaper formed as a substantially rectangular blank having tabs at the corners and elastic leg engaging areas to prevent or minimize leakage and to provide a secure and comfortable fit. For hygiene purposes the system includes a first and second storage compartment coextensive therewith for storing an integrated disposable bag and glove set for reducing feces and/or body fluid contamination during diaper removal. The system is configured with materials which provide a reduced overall package volume including both the bag and gloves.

Accordingly, it is a principal object of the invention to provide a disposable diaper system for removing soiled diapers from babies without feces and/or body fluid contamination.

It is another object of the invention to provide a disposable diaper system which is simple to use and deploy for diaper removal.

It is a further object of the invention to provide a disposable diaper system which eliminates waste with a reduced package volume, in accordance with OSHA (Occupational Safety and Health Administration) regulations.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a disposable diaper with bag according to the present invention.

FIG. 2 is an environmental, expanded perspective view of the disposable diaper according to the invention, illustrating disposable gloves and bag attachments thereon.

FIG. 3 is an elevational view of the disposable diaper according to the invention configured for disposal with the bag partly broken away to show the soiled diaper therein.

Similar reference characters denote corresponding features consistently throughout the attached drawings.

### DETAILED DESCRIPTION OF THE PREFERRED EMBODIMENT

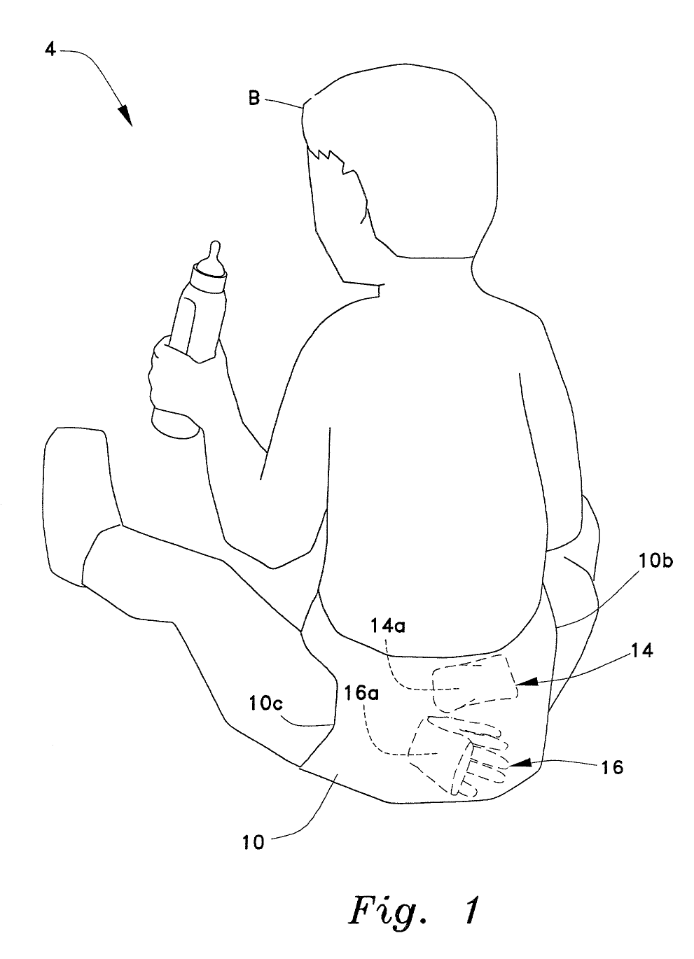

The present invention is directed to an improved disposable diaper system for infants, babies, senior citizens and/or the physically challenged B. The preferred embodiment of the present invention are depicted in FIGS. 1-3, and are generally referenced as numerals 4 in its configuration for use as a diaper and 5 in its configuration as a bag for disposal of the used diaper, respectively.

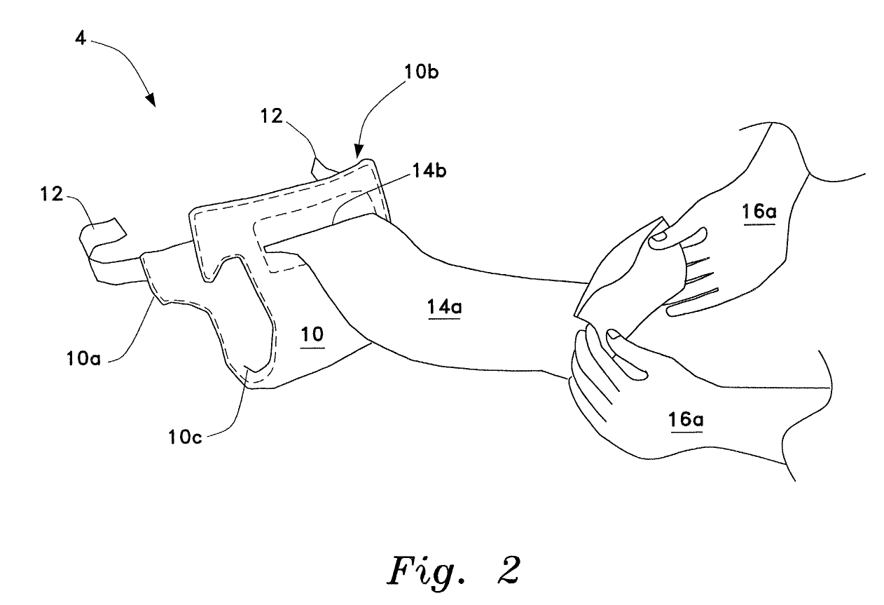

As best seen in FIG. 1, disposable diaper system 4 according to a first embodiment comprises a diaper 10 formed as a substantially rectangular blank having tabs 12 (as shown in FIG. 2) at the corners 10a and 10b, respectively. Adjacent to the corners 10a, 10b are elastic leg engaging areas 10c which prevent leakage and provide a secure and comfortable fit for the respective user B. The diaper system 4 further comprises respective first and second perforated storage compartments 14 and 16 coextensive therewith for storing a plurality integrated disposable elements, such as a bag 14a for disposal of the diaper and gloves 16a for reducing feces and/or body fluid contamination during diaper 10 removal. As better illustrated in FIG. 2, the bag 14a of the coextensive set is attached to or formed in one piece with the diaper 10 along an inner edge 14b of the perforated storage compartment 14 to reduce the overall package volume 18 of the removed soiled diaper 10′.

As recited above, the disposable diaper system 4 according to the invention includes therein the first and second storage compartments 14 and 16 which are accessed via peripheral perforations formed on an external surface portion of the diaper 10. It is preferred that compartments 14 and 16 be disposed upon the external rear surface portion of the disposable diaper 10 for easy and uninterruptable access by the person changing the diaper and to prevent unintended opening of the compartments 14 and 16 by the user B. In addition, it is preferred that the first and second storage compartments 14 and 16 be expandable storage compartments for fully deploying the bag 14a and expanding the glove or gloves 16a for use. This combination of bag 14a and gloves 16a with corresponding compartments 14 and 16, respectively, has a reduced package volume due to the use of a thin, water impervious plastic material. The bag 14a and glove 16a set can be sized according to a particular user's or care taker's physical or anatomical dimensions as a standardized dimensional feature.

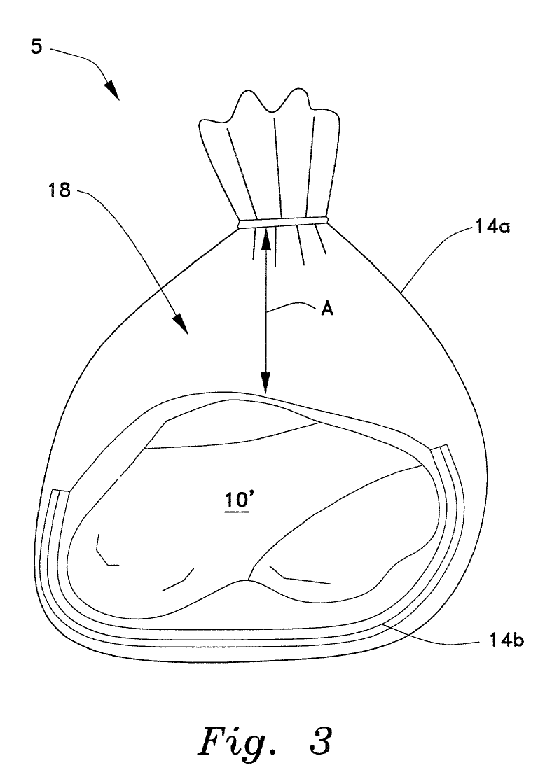

FIG. 3 shows the disposable diaper system 5 configured for disposal with a soiled diaper 10′ stored in the bag 14a (the disposable gloves 16a may also be placed in the bag 14a for disposal). The double head arrow A illustrates how the volume can be further reduced, as this particular area is simply captured ambient air. Other advantages of the disposable diaper system 4 and 5, include the use of light-weight multi-layered materials which have absorption characteristics which minimize leakage.

It is to be understood that the present invention is not limited to the sole embodiments described above, but encompasses any and all embodiments within the scope of the following claims.

We claim:

1. A disposable diaper system for infants, babies, senior citizens and/or those who are physically challenged, comprising:
a diaper formed as a substantially rectangular blank having tabs at the corners and elastic leg engaging areas to prevent leakage and to provide a secure and comfortable fit thereto, said diaper having a first and a second storage compartment defined therein, the first compartment having an inner edge;
a disposable bag stored in said first storage compartment, the bag being sized and dimensioned for containing said diaper for disposal and being attached to the inner edge of said first compartment; and
at least one disposable glove stored in said second storage compartment.

2. The disposable diaper system according to claim 1, wherein said first and second storage compartments are accessed via peripheral perforations formed on an external surface portion of the diaper.

3. The disposable diaper system according to claim 2, wherein said first and second storage compartments are disposed on the external rear surface portion of said diaper.

4. The disposable diaper system according to claim 1, wherein said first and second storage compartments are expandable storage compartments.
